(19) United States
(12) Patent Application Publication
(10) Pub. No.: US 2026/0260748 A1
(43) Pub. Date: Sep. 3, 2026

(54) SYSTEMS AND METHODS FOR IDENTIFYING MITOCHONDRIAL DNA VARIANTS

(71) Applicant: Foundation Medicine, Inc., Boston, MA (US)

(72) Inventors: Meagan Kathleen MONTESION, Somerville, MA (US); Julia HOPKINS, Cambridge, MA (US); Dexter X. JIN, Stoneham, MA (US); Zoe R. FLEISCHMANN, Jamaica Plain, MA (US)

(73) Assignee: Foundation Medicine, Inc., Boston, MA (US)

(21) Appl. No.: 19/159,586

(22) PCT Filed: Feb. 26, 2024
(86) PCT No.: PCT/US2024/017243

§ 371 Date: Aug. 25, 2025

Related U.S. Application Data
(60) Provisional application No. 63/448,555, filed on Feb. 27, 2023.

(51) Int. Cl. G16H 50/20; C12Q 1/6855; C12Q 1/6874; G16B 10/00; G16B 20/10; G16B 20/20; G16B 30/10; G16H 50/70

(52) U.S. Cl. CPC G16H 50/20; C12Q 1/6855; C12Q 1/6874; G16B 10/00; G16B 20/10; G16B 20/20; G16B 30/10; G16H 50/70; C12Q 2600/156

(57) ABSTRACT

Methods and systems for identifying mitochondrial DNA (mtDNA) variants are described. The methods may comprise, for example, receiving a plurality of off-target sequence reads obtained from a targeted sequencing method performed on a sample from a subject; aligning the plurality of off-target sequence reads to a mitochondrial reference genome to identify a plurality of mtDNA alignment reads; and processing the plurality of mtDNA alignment reads using a variant caller to identify a mtDNA variant.

### CROSS-REFERENCE TO RELATED APPLICATION

This application claims the priority benefit of U.S. Provisional Patent Application Ser. No. 63/448,555, filed Feb. 27, 2023, the contents of which are incorporated herein by reference in their entirety.

### FIELD OF THE DISCLOSURE

The present disclosure relates generally to methods and systems for the identification of DNA variants, and more specifically, to methods and systems for the identification of mitochondrial DNA (mtDNA) variants.

### BACKGROUND

Mitochondria are involved in major cellular processes. They are involved in metabolism, cell signaling, apoptosis, cell cycle, and cell proliferation. The dysfunction of mitochondria can have extensive effects on these cellular processes, and on the overall health of the subject or organism. In some cases, mitochondrial dysfunction can be the result of alterations in the mitochondrial genome, and can be associated with several different human diseases, including cancer. The identification of mitochondrial DNA (mtDNA) sequence variants and understanding their impact, however, is challenging. Improved methods are needed for identifying mtDNA sequence variants and determining their biological effects. The present disclosure addresses these needs.

### BRIEF SUMMARY OF THE INVENTION

Disclosed herein are methods and systems for identifying mtDNA variants. Existing methods for identifying mtDNA variants are often based on whole genome sequencing (WGS) or whole exome sequencing (WES). The methods disclosed herein employ off-target reads from a targeted sequencing method, such as, but not limited to, the next-generation sequencing of nuclear genomic content to identify mtDNA variants. The use of such off-target reads represent an innovative technique of salvaging data that may otherwise be discarded or unused. In addition, existing methods for identifying mtDNA variants can often require the use of a matched normal control. In other words, existing methods can often require a comparison sample derived from the same subject as an experimental sample. By comparing an experimental sample with a matched normal control derived from the same subject as the experimental sample, properties other than the biological property being investigated, such as mtDNA sequence variants, can be presumed to be otherwise similar or identical. In doing so, mtDNA sequence variants can be identified, because the variant sequences will differ from the matched normal control. The methods and systems disclosed herein address the above shortcomings seen in existing methods. The methods and systems of the present disclosure not only capitalize on off-target sequence reads obtained from a targeted sequencing method to identify mtDNA variants, but also do not require the use of a matched normal control to identify the mtDNA variants.

Disclosed herein are methods for identifying mtDNA variants, comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the capture nucleic acid molecules; receiving, at one or more processors, a plurality of off-target sequence reads obtained from a targeted sequencing method performed on the sample from the subject; aligning, using the one or more processors, the plurality of off-target sequence reads to a mitochondrial reference genome to identify a plurality of mtDNA alignment reads; and processing, using the one or more processors, the plurality of mtDNA alignment reads to identify a mtDNA variant.

In some embodiments, a variant caller is used to process the plurality of mtDNA alignment reads and identify the mtDNA variant. In some embodiments, the method can further comprise: determining, using the one or more processors, an allele frequency for the mtDNA variant based on the plurality of mtDNA alignment reads; and categorizing, using the one or more processors, the mtDNA variant as germline, somatic, or as subclonal or an artifact, based on the determined allele frequency. In some embodiments, the categorization does not require use of sequence read data from a matched normal control.

In any of the embodiments herein, the subject can be suspected of having or is determined to have cancer. In some embodiments, the cancer can be a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer can comprise acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the methods disclosed herein can further comprise treating the subject with an anti-cancer therapy. In some embodiments, the anti-cancer therapy can comprise a targeted anti-cancer therapy. In some embodiments, the targeted anti-cancer therapy can comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Pigray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In any of the embodiments herein, the methods can further comprise obtaining the sample from the subject. In any of the embodiments herein, the sample comprises a tissue biopsy sample, a liquid biopsy sample, or a normal control. In some embodiments, the sample can be a liquid biopsy sample and can comprise blood, plasma, cerebrospinal fluid, sputum, stool, urine, or saliva. In some embodiments, the sample can be a liquid biopsy sample and can comprise circulating tumor cells (CTCs). In some embodiments, the sample can be a liquid biopsy sample and comprises cell-free DNA (cfDNA), circulating tumor DNA (ctDNA), or any combination thereof. In any of the embodiments herein, the plurality of nucleic acid molecules can comprise a mixture of tumor nucleic acid molecules and non-tumor nucleic acid molecules. In some embodiments, the tumor nucleic acid molecules can be derived from a tumor portion of a heterogeneous tissue biopsy sample, and the non-tumor nucleic acid molecules are derived from a normal portion of the heterogeneous tissue biopsy sample. In some embodiments, the sample can comprise a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In any of the embodiments herein, the one or more adapters can comprise amplification primers, flow cell adaptor sequences, substrate adapter sequences, or sample index sequences. In any of the embodiments herein, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule.

In any of the embodiments herein, amplifying nucleic acid molecules can comprise performing a polymerase chain reaction (PCR) amplification technique, a non-PCR amplification technique, or an isothermal amplification technique. In any of the embodiments herein, the sequencing can comprise use of a massively parallel sequencing (MPS) technique, whole genome sequencing (WGS), whole exome sequencing, targeted sequencing, direct sequencing, or Sanger sequencing technique. In some embodiments, the sequencing can comprise massively parallel sequencing, and the massively parallel sequencing technique comprises next generation sequencing (NGS). In any of the embodiments herein, the sequencer can comprise a next generation sequencer.

In any of the embodiments herein, one or more of the plurality of sequencing reads can overlap one or more gene loci within one or more subgenomic intervals in the sample. In some embodiments, the one or more gene loci can comprise between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In any of the embodiments herein, the one or more gene loci can comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70,CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1,CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4(C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In any of the embodiments herein, the one or more gene loci can comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof.

In any of the embodiments herein, the methods can further comprise generating, by the one or more processors, a report indicating the presence or absence of the genetic variant. In some embodiments, the methods can further comprise transmitting the report to a healthcare provider. In some embodiments, the report can be transmitted via a computer network or a peer-to-peer connection.

Disclosed herein is a method for identifying mtDNA variants, comprising: receiving, at one or more processors, a plurality of off-target sequence reads obtained from a targeted sequencing method performed on a sample from a subject; aligning, using the one or more processors, the plurality of off-target sequence reads toa mitochondrial reference genome to identify a plurality of mtDNA alignment reads; and processing, using the one or more processors, the plurality of mtDNA alignment reads to identify a mtDNA variant.

In some embodiments, a variant caller is used to process the plurality of mtDNA alignment reads and identify the mtDNA variant. In some embodiments, the method can further comprise: determining, using the one or more processors, an allele frequency for the mtDNA variant based on the plurality of mtDNA alignment reads; and categorizing, using the one or more processors, the mtDNA variant as germline, somatic, or as subclonal or an artifact, based on the determined allele frequency. In some embodiments, the categorization does not require use of sequence read data from a matched normal control. In any of the embodiments herein, the off-target sequence reads can be identified by aligning sequence reads from the targeted sequencing method to a non-mitochondrial reference genome. In some embodiments, the non-mitochondrial reference genome can be a human reference genome. In some embodiments, the human reference genome can be hg19. In any of the embodiments herein, the off-target sequence reads can comprise sequence reads that align to a mitochondrial genome and sequence reads that do not align to a non-mitochondrial reference genome.

In some aspects, disclosed herein is a method for identifying mtDNA variants, comprising: a) receiving, at one or more processors, a plurality of sequence reads obtained from a targeted sequencing method performed on a sample from a subject; aligning, using the one or more processors, the plurality of sequence reads to a mitochondrial reference genome, to identify a plurality of mtDNA alignment reads; processing, using the one or more processors, the plurality of mtDNA alignment reads to identify a mtDNA variant; determining, using the one or more processors, an allele frequency for the mtDNA variant based on the plurality of mtDNA alignment reads; and categorizing, using the one or more processors, the mtDNA variant as germline, somatic, or as subclonal or an artifact, based on the determined allele frequency, wherein the categorization does not require use of sequence read data from a matched normal control.

In any of the embodiments herein, a variant caller may be used to process the plurality of mtDNA alignment reads and identify the mtDNA variant. In any of the embodiments herein, the mitochondrial reference genome can be the revised Cambridge Reference Sequence. In any of the embodiments herein, the variant caller can comprise a short variant caller. In any of the embodiments herein, the variant caller can comprise a mtDNA rearrangement caller. In any of the embodiments herein, the variant caller can comprise a large mtDNA deletion caller.

In any of the embodiments herein, a mtDNA variant can be classified as germline if its allele frequency is greater than or equal to a first threshold. In some embodiments, the first threshold can be about 90%, 95% or 98%. In any of the embodiments herein, a mtDNA variant can be classified as somatic if its allele frequency is less than the first threshold and greater than or equal to a second threshold. In some embodiments, the second threshold can be about 2%, 5%, or 10%. In any of the embodiments herein, a mtDNA variant is classified as subclonal or as an artifact if its allele frequency is less than the second threshold. In some embodiments, the first threshold can be about 95%, and the second threshold can be about 5%.

In any of the embodiments herein mtDNA variant allele frequencies can be used to identify a mitochondrial haplogroup. In some embodiments, the mitochondrial haplogroup can be used to determine ancestral data regarding the subject.

In some aspects, disclosed herein is a method for detection of a disease in a subject, the method comprising: a) receiving, at one or more processors, a plurality of off-target sequence reads obtained from a targeted sequencing method performed on a sample from the subject; b) aligning, using the one or more processors, the plurality of sequence reads to a mitochondrial reference genome to identify a plurality of mtDNA alignment reads; c) processing, using the one or more processors, the plurality of mtDNA alignment reads using a variant caller to identify a mtDNA variant; and d) detecting, using the one or more processors, a disease signal in the subject based on at least one identified mtDNA variant, wherein the disease signal is indicative of a disease status of the subject.

In some embodiments, the disease can comprise a thyroid cancer and the mtDNA variant is a G3244A substitution in a mitochondrial leucine tRNA (MT-TL1) gene. In some embodiments, the thyroid cancer can be Hurthle cell carcinoma. In some embodiments, the disease can comprise mitochondrial encephalomyopathy, lactic acidosis, and stroke-like episodes (MELAS) and the mtDNA variant can be a G3244A substitution. In some embodiments, the disease can comprise a thyroid cancer and the mtDNA variant can be a G3380A substitution in a NADH dehydrogenase (ND1) gene. In some embodiments, the thyroid cancer can be thyroid anaplastic carcinoma. In some embodiments, the disease can comprise a colorectal cancer and the mtDNA variant can be a G3380A substitution in a NADH dehydrogenase (ND1) gene. In some embodiments, the disease can comprise MELAS and the mtDNA variant is a G3380A substitution.

In any of the embodiments herein, the subject can be a human. In any of the embodiments herein, the sample can comprise a tissue biopsy sample. In any of the embodiments herein, the sample can comprise a liquid biopsy sample. In any of the embodiments herein, the liquid biopsy sample can comprise blood, plasma, cerebrospinal fluid, sputum, stool, urine, or saliva.

Disclosed herein are methods comprising diagnosing a disease based on an identification of a mtDNA variant for a sample from the subject, wherein the mtDNA variant is determined according to the methods disclosed herein. Disclosed herein is a method of selecting an anti-cancer therapy, the method comprising: responsive to identifying a mtDNA variant for a sample from a subject, selecting an anti-cancer therapy for the subject, wherein identifying a mtDNA variant is determined according to the methods disclosed herein. Disclosed herein is a method of treating a cancer in a subject, comprising: responsive to identifying a mtDNA variant for a sample from the subject, administering an effective amount of an anti-cancer therapy to the subject, wherein identifying a mtDNA variant is determined according to the methods disclosed herein. Disclosed herein is a method for monitoring cancer progression or recurrence in a subject, the method comprising: determining a first identification of a mtDNA variant in a first sample obtained from the subject at a first time point according to the methods disclosed herein; determining a second identification of a mtDNA variant in a second sample obtained from the subject at a second time point; and comparing the first mtDNA variant to the second mtDNA variant, thereby monitoring the cancer progression or recurrence. In some embodiments, the second mtDNA variant for the second sample is determined according to the methods disclosed herein. In some embodiments, the methods disclosed herein can further comprise selecting an anti-cancer therapy for the subject in response to the cancer progression. In any of the embodiments herein, the methods disclosed herein can further comprise administering an anti-cancer therapy to the subject in response to the cancer progression. In any of the embodiments herein, the methods of the present disclosure can further comprise adjusting an anti-cancer therapy for the subject in response to the cancer progression. In any of the embodiments herein, the methods of the present disclosure can further comprise adjusting a dosage of the anti-cancer therapy or selecting a different anti-cancer therapy in response to the cancer progression. In some embodiments, the methods of the present disclosure can further comprise administering the adjusted anti-cancer therapy to the subject. In any of the embodiments herein, the first time point is before the subject has been administered an anti-cancer therapy, and wherein the second time point is after the subject has been administered the anti-cancer therapy. In any of the embodiments herein, the subject has a cancer, is at risk of having a cancer, is being routine tested for cancer, or is suspected of having a cancer. In any of the embodiments herein, the cancer is a solid tumor. In any of the embodiments herein, the cancer is a hematological cancer.

In any of the embodiments herein, the anti-cancer therapy can comprise chemotherapy, radiation therapy, immunotherapy, a targeted therapy, or surgery. In any of the embodiments herein, the methods disclosed herein can further comprise determining, identifying, or applying the mtDNA variant for the sample as a diagnostic value associated with the sample. In any of the embodiments herein, the methods of the present disclosure can further comprise generating a genomic profile for the subject based on the identification of a mtDNA variant. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In any of the embodiments herein, the genomic profile for the subject further comprises results from a nucleic acid sequencing-based test. In any of the embodiments herein, the methods of the present disclosure can further comprise selecting an anti-cancer therapy, administering an anti-cancer therapy, or applying an anti-cancer therapy to the subject based on the generated genomic profile. In any of the embodiments herein, the identification of an mtDNA variant for the sample is used in making suggested treatment decisions for the subject. In any of the embodiments herein, the identification of an mtDNA variant for the sample is used in applying or administering a treatment to the subject.

In some aspects, disclosed herein is a system comprising: a) one or more processors; and b) a memory communicatively coupled to the one or more processors and configured to store instructions that, when executed by the one or more processors, cause the system to: i) receive a plurality of off-target sequence reads obtained from a targeted sequencing method performed on a sample from a subject; ii) align the plurality of off-target sequence reads to a mitochondrial reference genome to identify a plurality of mtDNA alignment reads; and iii) process the plurality of mtDNA alignment reads to identify a mtDNA variant. In some embodiments, a variant caller is used to process the plurality of mtDNA alignment reads and identify the mtDNA variant. In some embodiments, the off-target sequence read data can be identified by aligning sequence reads from a targeted sequencing method to a non-mitochondrial reference genome. In some embodiments, the off-target sequence reads can comprise sequence reads that align to a mitochondrial genome and sequence reads that do not align to a non-mitochondrial reference genome. In any of the embodiments herein, the variant caller can comprise a short variant caller. In any of the embodiments herein, the system can further comprise instructions that, when executed by the one or more processors, cause the system to: c) determine an allele frequency for the mtDNA variant based on the plurality of mtDNA alignment reads; and d) categorize the mtDNA variant as germline, somatic, or as subclonal or an artifact, based on the determined allele frequency. In some embodiments, the categorization does not require use of sequence read data from a matched normal control.

In some aspects, disclosed herein is a non-transitory computer-readable storage medium storing one or more programs, the one or more programs comprising instructions, which when executed by one or more processors of a system, can cause the system to: a) receive a plurality of off-target sequence reads obtained from a targeted sequencing method performed on a sample from a subject; b) align the plurality of sequence reads to a mitochondrial reference genome to identify a plurality of mtDNA alignment reads; and c) process the plurality of mtDNA alignment reads to identify a mtDNA variant. In some embodiments, a variant caller is used to process the plurality of mtDNA alignment reads and identify the mtDNA variant. In some embodiments, the off-target sequence reads can be identified by aligning sequence reads from a targeted sequencing method to a non-mitochondrial reference genome. In some embodiments, the off-target sequence reads can comprise sequence reads that align to a mitochondrial genome and sequence reads that do not align to a non-mitochondrial reference genome. In some embodiments, the variant caller can comprise a short variant caller. In some embodiments, the non-transitory computer-readable storage medium can further comprise instructions that, when executed by the one or more processors, can cause the system to: d) determine an allele frequency for the mtDNA variant based on the plurality of mtDNA alignment reads; and e) categorize the mtDNA variant as germline, somatic, or as subclonal or an artifact, based on the determined allele frequency. In some embodiments, the categorization does not require use of sequence read data from a matched normal control.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 depicts a non-limiting exemplary method for identifying mtDNA variants, in accordance with some embodiments of the present disclosure.

FIG. 2 depicts an additional non-limiting exemplary method for identifying mtDNA variants, in accordance with some embodiments of the present disclosure.

FIG. 3 depicts an exemplary computing device or system, in accordance with some embodiments of the present disclosure.

FIG. 4 depicts an exemplary computer system or computer network, in accordance with some instances of the systems described herein.

FIG. 5 depicts a non-limiting example of data that illustrates a histogram of mtDNA allele frequencies, in accordance with some embodiments of the present disclosure.

FIG. 6 depicts a non-limiting example of data that illustrates a histogram of mtDNA allele frequencies, in accordance with some embodiments of the present disclosure.

FIG. 7 depicts a non-limiting example of data that illustrates mitochondrial haplogroups, in accordance with some embodiments of the present disclosure.

FIGS. 8A-B depict non-limiting examples of data that illustrates statistics relating diseases to mtDNA variants, in accordance with some embodiments of the present disclosure.

FIG. 9 depicts a non-limiting exemplary schematic that illustrates a portion of a mtDNA sequence, in accordance with some embodiments of the present disclosure.

FIG. 10 depicts a non-limiting example of data that illustrates the prevalence of at least one mtDNA variant across disease types, in accordance with some embodiments of the present disclosure.

FIG. 11 depicts a non-limiting example of data that illustrates the relationship between at least one mtDNA variant and disease types, in accordance with some embodiments of the present disclosure.

FIG. 12 depicts a non-limiting example of data that illustrates the relationship between at least one mtDNA variant and disease types, in accordance with some embodiments of the present disclosure.

### DETAILED DESCRIPTION

Disclosed herein are methods and systems for identifying mtDNA variants. Existing methods for identifying mtDNA variants are often based on whole genome sequencing (WGS) or whole exome sequencing (WES). The methods disclosed herein employ off-target reads from a targeted sequencing method, such as, but not limited to, the next-generation sequencing of nuclear genomic content to identify mtDNA variants. The use of such off-target reads represent an innovative technique of salvaging data that may otherwise be discarded or unused. In addition, existing methods for identifying mtDNA variants can often require the use of a matched normal control. In other words, existing methods can often require a comparison sample derived from the same subject as an experimental sample. By comparing an experimental sample with a matched normal control derived from the same subject as the experimental sample, properties other an the biological property being investigated, such as mtDNA sequence variants, can be presumed to be otherwise similar, if not identical. In doing so, mtDNA sequence variants can be identified, because the variant sequences will differ from the matched normal control. The methods and systems disclosed herein address the above shortcomings seen in existing methods. The methods and systems of the present disclosure not only capitalize on off-target sequence reads obtained from a targeted sequencing method to identify mtDNA variants, but also do not require the use of a matched normal control to identify the mtDNA variants.

In some instances, for example, the disclosed methods for identifying mtDNA variants can comprise: a) receiving a plurality of off-target sequence reads obtained from a targeted sequencing method performed on a sample from a subject; b) aligning the plurality of off-target sequence reads to a mitochondrial reference genome to identify a plurality of mtDNA alignment reads; and c) processing the plurality of mtDNA alignment reads using a variant caller to identify a mtDNA variant.

In some instances, the method may further comprise: d) determining an allele frequency for the mtDNA variant based on the plurality of mtDNA alignment reads; and e) categorizing the mtDNA variant as germline, somatic, or as subclonal or an artifact, based on the determined allele frequency.

In some instances, for example, the disclosed methods for identifying mtDNA variants can comprise: a) receiving a plurality of sequence reads obtained from a targeted sequencing method performed on a sample from a subject; b) aligning the plurality of sequence reads to a mitochondrial reference genome, to identify a plurality of mtDNA alignment reads; c) processing the plurality of mtDNA alignment reads using a variant caller to identify a mtDNA variant; d) determining an allele frequency for the mtDNA variant based on the plurality of mtDNA alignment reads; and e) categorizing the mtDNA variant as germline, somatic, or as subclonal or an artifact, based on the determined allele frequency, wherein the categorization does not require use of sequence read data from a matched normal control.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”') refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Identifying mtDNA Variants

Mitochondria are eukaryotic organelles that are responsible for producing energy for cells, and are involved with major cellular processes including metabolism, signaling, apoptosis, cell cycle, and proliferation as noted above. Each cell can contain up to thousands of mitochondria, each of which contain multiple copies of its own genome (mtDNA or chrM) that is roughly 16 kb in length and encodes 37 genes. Mitochondrial dysfunction, which can be due to genomic alterations, among other mechanisms, is associated with several human diseases, including cancer.

Due to the high amount of mtDNA in each cell, the methods of the present disclosure seek to utilize off-target next-generation sequencing reads to identify mtDNA short variant alterations. All chrM-mapped and hg19-unmapped reads were extracted from the BAM files created by a next-generation sequencing pipeline. These sequence reads were then realigned to the Revised Cambridge Reference Sequence (rCRS) of human mitochondrial DNA to create a new mtDNA-specific BAM file. This BAM file was then processed using, e.g., a short variant (SV) caller to identify mtDNA short variants and, in some instances, to calculate allele frequency. Allele frequency (AF) was used to estimate whether a mutation was likely to be somatic or germline.

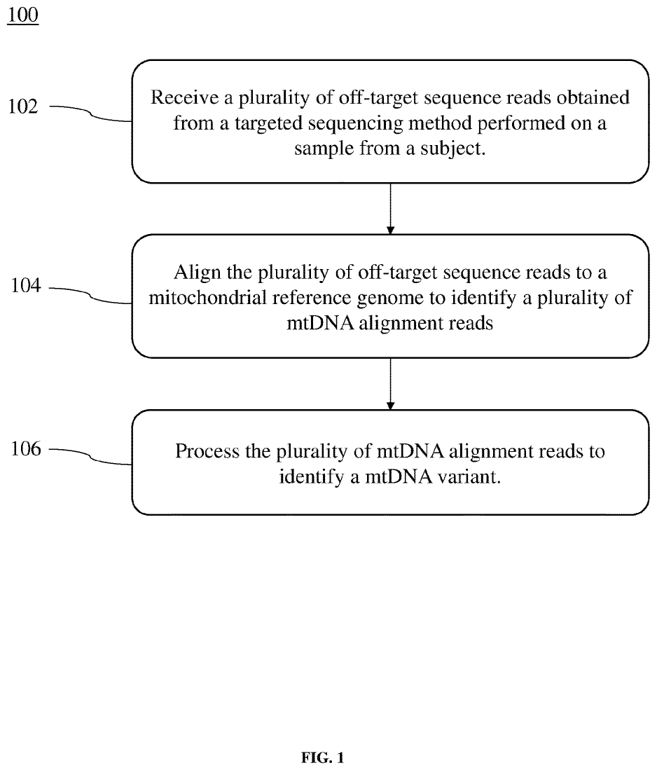

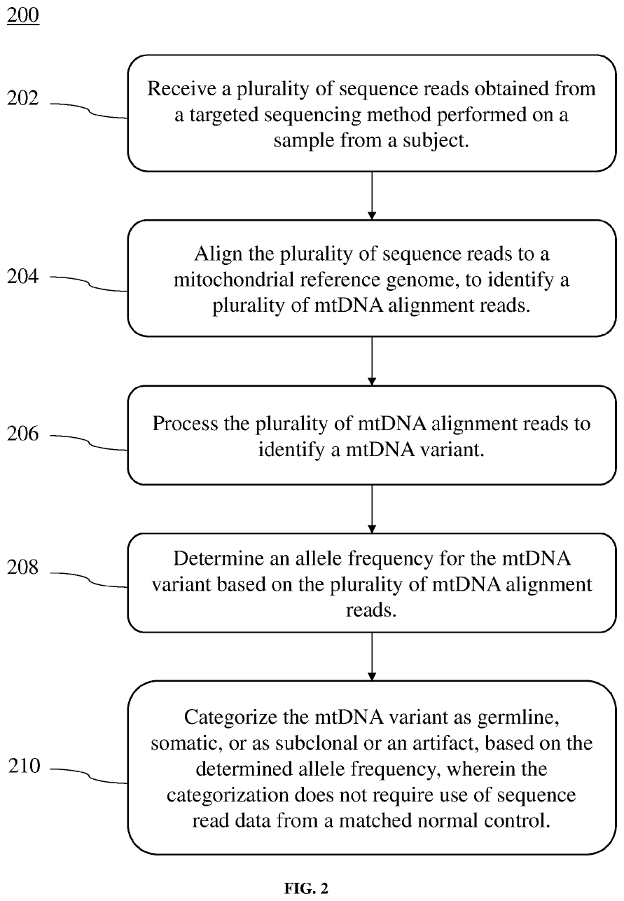

FIG. 1 illustrates an exemplary schematic showing a general process 100 for identifying mtDNA variants. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

In some aspects, disclosed herein is a method identifying mtDNA variants comprising: A method for identifying mitochondrial DNA (mtDNA) variants, comprising: a) receiving, at one or more processors, a plurality of off-target sequence reads obtained from a targeted sequencing method performed on a sample from a subject; b) aligning, using the one or more processors, the plurality of off-target sequence reads to a mitochondrial reference genome to identify a plurality of mtDNA alignment reads; and c) processing, using the one or more processors, the plurality of mtDNA alignment reads using a variant caller to identify a mtDNA variant. In some aspects, disclosed herein is a method further comprising: d) determining, using the one or more processors, an allele frequency for the mtDNA variant based on the plurality of mtDNA alignment reads; and categorizing, using the one or more processors, the mtDNA variant as germline, somatic, or as subclonal or an artifact, based on the determined allele frequency.

At step 102 in FIG. 1, a plurality of off-target sequence reads obtained from a targeted sequencing method performed on a sample from a subject is received.

In any of the instances described herein, the off-target sequence reads can be identified by aligning sequence reads from the targeted sequencing method to a non-mitochondrial reference genome. In some instances, the non-mitochondrial reference genome can be a human reference genome. In some instances, the human reference genome can be hg19. In any of the instances herein, the off-target sequence reads can comprise sequence reads that align to a mitochondrial genome and sequence reads that do not align to a non-mitochondrial reference genome.

At step 104 in FIG. 1, the plurality of off-target sequence reads are aligned to a mitochondrial reference genome to identify a plurality of mtDNA alignment reads.

In any of the instances described herein, the mitochondrial reference genome can be the revised Cambridge Reference Sequence.

At step 106 in FIG. 1, the plurality of mtDNA alignment reads is processed, using a variant caller to identify a mtDNA variant.

In any of the instances described herein, the variant caller can comprise, e.g., a short variant caller. In any of the instances described herein, the variant caller can comprise a mtDNA rearrangement caller. In any of the instances described herein, the variant caller can comprise a large mtDNA deletion caller.

The steps depicted in FIG. 1 can be supplemented by additional steps. For example, in some instances an allele frequency for the mtDNA variant based on the plurality of mtDNA alignment reads can be determined.

In some instances, mtDNA variant allele frequencies can be used to identify a mitochondrial haplogroup. In some instances, the mitochondrial haplogroup can be used to determine ancestral data regarding the subject.

In some instances, allele frequencies can be calculated by computing the fraction of the members of a set that exhibit a given allele, such as, but not limited to, the fraction of cells of a sample that exhibit the given allele. In some instances, the allele frequencies of mtDNA variants can comprise the fraction of genomic copies within a single cell that exhibit the allele, because each cell can comprise tens to thousands of copies of the mitochondrial genome. Similarly, in some instances, the allele frequencies of mtDNA variants can comprise the fraction of cells of a sample that exhibit given alleles, wherein each cell can comprise tens to thousands of copies of the mitochondrial genome.

In some instances, an algorithm, such as, but not limited to, an open-source algorithm (e.g., MtoolBox), can be used to identify haplogroups based, at least in part, on mtDNA variant sequences. The algorithm can be used to identify the haplogroup that has the same polymorphisms as those present in the mtDNA sequences from a sample. In doing so, mtDNA variants can be classified into at least one haplogroup with varying probabilities or confidence scores.

The steps depicted in FIG. 1 can be supplemented by additional steps. For example, the mtDNA variant can be categorized as germline, somatic, or as subclonal or an artifact, based on the determined allele frequency.

In some instances, the categorization of the mtDNA variant does not require use of sequence read data from a matched normal control.

In any of the instances described herein, a mtDNA variant can be classified as germline if its allele frequency is greater than or equal to a first threshold. In some instances, the first threshold can be about 90%, 95% or 98%. In any of the instances described herein, a mtDNA variant can be classified as somatic if its allele frequency is less than the first threshold and greater than or equal to a second threshold. In some instances, the second threshold can be about 2%, 5%, or 10%. In any of the instances described herein, a mtDNA variant is classified as subclonal or as an artifact if its allele frequency is less than the second threshold. In some instances, the first threshold can be about 95%, and the second threshold can be about 5%.

FIG. 2 illustrates an exemplary schematic showing a general process 200 for identifying mtDNA variants. Process 200 can be performed, for example, using one or more electronic devices implementing a software platform. In some examples, process 200 is performed using a client-server system, and the blocks of process 200 are divided up in any manner between the server and a client device. In other examples, the blocks of process 200 are divided up between the server and multiple client devices. Thus, while portions of process 200 are described herein as being performed by particular devices of a client-server system, it will be appreciated that process 200 is not so limited. In other examples, process 200 is performed using only a client device or only multiple client devices. In process 200, some blocks are, optionally, combined, the order of some blocks is, optionally, changed, and some blocks are, optionally, omitted. In some examples, additional steps may be performed in combination with the process 200. Accordingly, the operations as illustrated (and described in greater detail below) are exemplary by nature and, as such, should not be viewed as limiting.

In some aspects, disclosed herein is a method for identifying mtDNA variants, comprising: a) receiving, at one or more processors, a plurality of sequence reads obtained from a targeted sequencing method performed on a sample from a subject; b) aligning, using the one or more processors, the plurality of sequence reads to a mitochondrial reference genome, to identify a plurality of mtDNA alignment reads; c) processing, using the one or more processors, the plurality of mtDNA alignment reads using a variant caller to identify a mtDNA variant; d) determining, using the one or more processors, an allele frequency for the mtDNA variant based on the plurality of mtDNA alignment reads; and e) categorizing, using the one or more processors, the mtDNA variant as germline, somatic, or as subclonal or an artifact, based on the determined allele frequency, wherein the categorization does not require use of sequence read data from a matched normal control.

At step 202 in FIG. 2, a plurality of sequence reads obtained from a targeted sequencing method performed on a sample from a subject is received.

In any of the instances described herein, the sequence reads can be identified by aligning sequence reads from the targeted sequencing method to a non-mitochondrial reference genome. In some instances, the non-mitochondrial reference genome can be a human reference genome. In some instances, the human reference genome can be hg19. In any of the instances herein, the sequence reads can comprise sequence reads that align to a mitochondrial genome and sequence reads that do not align to a non-mitochondrial reference genome.

At step 204 in FIG. 2, the plurality of off-target sequence reads are aligned to a mitochondrial reference genome to identify a plurality of mtDNA alignment reads.

In any of the instances described herein, the mitochondrial reference genome can be the revised Cambridge Reference Sequence.

At step 206 in FIG. 2, the plurality of mtDNA alignment reads is processed, using a variant caller to identify a mtDNA variant.

In any of the instances described herein, the variant caller can comprise, e.g., a short variant caller. In any of the instances described herein, the variant caller can comprise a mtDNA rearrangement caller. In any of the instances described herein, the variant caller can comprise a large mtDNA deletion caller.

At step 208 in FIG. 2, an allele frequency for the mtDNA variant based on the plurality of mtDNA alignment reads is determined.

In some instances, mtDNA variant allele frequencies can be used to identify a mitochondrial haplogroup. In some instances, the mitochondrial haplogroup can be used to determine ancestral data regarding the subject.

In some instances, allele frequencies can be calculated by computing the fraction of the members of a set that exhibit a given allele, such as, but not limited to, the fraction of cells of a sample that exhibit the given allele. In some instances, the allele frequencies of mtDNA variants can comprise the fraction of genomic copies within a single cell that exhibit the allele, because each cell can comprise tens to thousands of copies of the mitochondrial genome. Similarly, in some instances, the allele frequencies of mtDNA variants can comprise the fraction of cells of a sample that exhibit given alleles, wherein each cell can comprise tens to thousands of copies of the mitochondrial genome.

In some instances, an algorithm, such as, but not limited to, an open-source algorithm (e.g., MtoolBox), can be used to identify haplogroups based, at least in part, on mtDNA variant sequences. The algorithm can be used to identify the haplogroup that has the same polymorphisms as those present in the mtDNA sequences from a sample. In doing so, mtDNA variants can be classified into at least one haplogroup with varying probabilities or confidence scores.

At step 210 in FIG. 2, the mtDNA variant is categorized as germline, somatic, or as subclonal or an artifact, based on the determined allele frequency, wherein the categorization does not require use of sequence read data from a matched normal control.

In some instances, the categorization of the mtDNA variant does not require use of sequence read data from a matched normal control.

In any of the instances described herein, a mtDNA variant can be classified as germline if its allele frequency is greater than or equal to a first threshold. In some instances, the first threshold can be about 90%, 95% or 98%. In any of the instances described herein, a mtDNA variant can be classified as somatic if its allele frequency is less than the first threshold and greater than or equal to a second threshold. In some instances, the second threshold can be about 2%, 5%, or 10%. In any of the instances described herein, a mtDNA variant is classified as subclonal or as an artifact if its allele frequency is less than the second threshold. In some instances, the first threshold can be about 95%, and the second threshold can be about 5%.

In some aspects, disclosed herein is a method for detection of a disease in a subject, the method comprising: a) receiving, at one or more processors, a plurality of off-target sequence reads obtained from a targeted sequencing method performed on a sample from the subject; b) aligning, using the one or more processors, the plurality of sequence reads to a mitochondrial reference genome to identify a plurality of mtDNA alignment reads; c) processing, using the one or more processors, the plurality of mtDNA alignment reads using a variant caller to identify a mtDNA variant; and d) detecting, using the one or more processors, a disease signal in the subject based on at least one identified mtDNA variant, wherein the disease signal is indicative of a disease status of the subject.

In some instances, the disease can comprise a thyroid cancer and the mtDNA variant is a G3244A substitution in a mitochondrial leucine tRNA (MT-TL1) gene. In some instances, the thyroid cancer can be Hurthle cell carcinoma. In some instances, the disease can comprise mitochondrial encephalomyopathy, lactic acidosis, and stroke-like episodes (MELAS) and the mtDNA variant can be a G3244A substitution. In some instances, the disease can comprise a thyroid cancer and the mtDNA variant can be a G3380A substitution in a NADH dehydrogenase (ND1) gene. In some instances, the thyroid cancer can be thyroid anaplastic carcinoma. In some instances, the disease can comprise a colorectal cancer and the mtDNA variant can be a G3380A substitution in a NADH dehydrogenase (ND1) gene. In some instances, the disease can comprise MELAS and the mtDNA variant is a G3380A substitution.

In any of the instances herein, the subject can be a human. In any of the instances herein, the sample can comprise a tissue biopsy sample. In any of the instances herein, the sample can comprise a liquid biopsy sample. In any of the instances herein, the liquid biopsy sample can comprise blood, plasma, cerebrospinal fluid, sputum, stool, urine, or saliva.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (viii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for identifying mtDNA variants may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for identifying mtDNA variants may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for identifying mtDNA variants may be used to select a subject (e.g., a patient) for a clinical trial based on the identified mtDNA variants. In some instances, patient selection for clinical trials based on, e.g., identification of specific mtDNA variants at one or more loci, may accelerate the development of targeted therapies and improve the healthcare outcomes for treatment decisions.

In some instances, the disclosed methods for identifying mtDNA variants may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for identifying mtDNA variants may be used in treating a disease (e.g., a cancer) in a subject. For example, in response to identifying mtDNA variants using any of the methods disclosed herein, an effective amount of an anti-cancer therapy or anti-cancer treatment may be administered to the subject.

In some instances, the disclosed methods for identifying a mtDNA variant may be used for monitoring disease progression or recurrence (e.g., cancer or tumor progression or recurrence) in a subject. For example, in some instances, the methods may be used to identify a mtDNA variant in a first sample obtained from the subject at a first time point, and used to a mtDNA variant in a second sample obtained from the subject at a second time point, where comparison of the first identification of a mtDNA variant and the second identification of a mtDNA variant allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the identification of a mtDNA variant In some instances, the presence of one or more mtDNA variants identified using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for identifying a mtDNA variant may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for identifying a mtDNA variant as part of a genomic profiling process (or inclusion of the output from the disclosed methods for identifying a mtDNA variant as part of the genomic profile of the subject) can improve the validity of, e.g., disease detection calls and treatment decisions, made on the basis of the genomic profile by, for example, independently confirming the presence of one or more mtDNA variants in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells.

Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e. g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e. g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB 351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques.

The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1): 35-42; Masuda, et al., (1999) Nucleic Acids Res. 27(22): 4436-4443; Specht, et al., (2001) Am J Pathol. 158(2): 419-429; the Ambion RecoverAlI™ Total Nucleic Acid Isolation Protocol (Ambion, Cat. No. AM1975, September 2008); the Maxwell® 16 FFPE Plus LEV DNA Purification Kit Technical Manual (Promega Literature #TM 349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAlI™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Target Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D.T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No.

WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1 e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9): 1297-303; Ye, K., et al., Bioinformatics, 2009; 25(21): 2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011;21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011;21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 March 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e. g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Systems

Also disclosed herein are systems designed to implement any of the disclosed methods for identifying a mtDNA variant in a sample from a subject. The systems may comprise, e.g., one or more processors, and a memory unit communicatively coupled to the one or more processors and configured to store instructions that, when executed by the one or more processors, cause the system to: i) receive a plurality of off-target sequence reads obtained from a targeted sequencing method performed on a sample from a subject; ii) align the plurality of off-target sequence reads to a mitochondrial reference genome to identify a plurality of mtDNA alignment reads; and iii) process the plurality of mtDNA alignment reads using a variant caller to identify a mtDNA variant.

In some instances, the off-target sequence read data can be identified by aligning sequence reads from a targeted sequencing method to a non-mitochondrial reference genome. In some instances, the off-target sequence reads can comprise sequence reads that align to a mitochondrial genome and sequence reads that do not align to a non-mitochondrial reference genome. In any of the instances herein, the variant caller can comprise a short variant caller. In any of the instances herein, the system can further comprise instructions that, when executed by the one or more processors, cause the system to: determine an allele frequency for the mtDNA variant based on the plurality of mtDNA alignment reads; and categorize the mtDNA variant as germline, somatic, or as subclonal or an artifact, based on the determined allele frequency. In some instances, the categorization does not require use of sequence read data from a matched normal control.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for identifying a mtDNA variant in any of a variety of samples as described herein (e.g., a tissue sample, biopsy sample, hematological sample, or liquid biopsy sample derived from the subject).

In some instances, the sequencing data may be processed to identify a mtDNA variant at at least 0, 1, 2, 3, 4, 5, 6, 7, 8, 9, 10, or more than 10 chrM loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the identification of a mtDNA variant is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

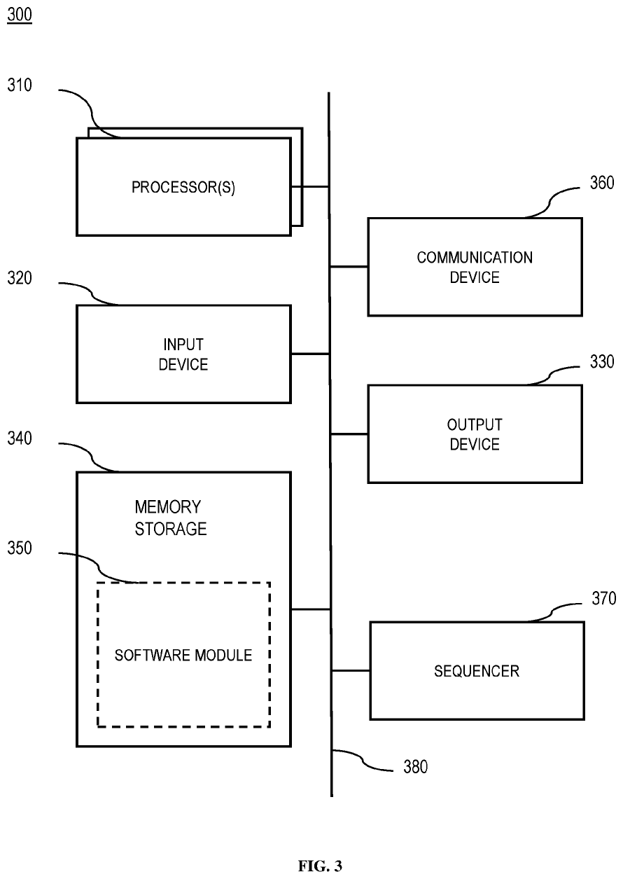

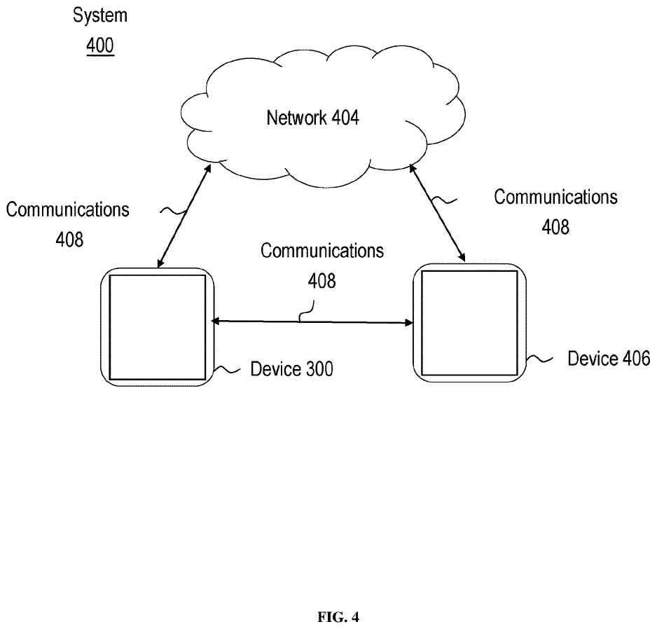

FIG. 3 illustrates an example of a computing device or system in accordance with one embodiment. Device 300 can be a host computer connected to a network. Device 300 can be a client computer or a server. As shown in FIG. 3, device 300 can be any suitable type of microprocessor-based device, such as a personal computer, workstation, server or handheld computing device (portable electronic device) such as a phone or tablet. The device can include, for example, one or more processor(s) 310, input devices 320, output devices 330, memory or storage devices 340, communication devices 360, and nucleic acid sequencers 370. Software 350 residing in memory or storage device 340 may comprise, e.g., an operating system as well as software for executing the methods described herein. Input device 320 and output device 330 can generally correspond to those described herein, and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage (e.g., an electrical, magnetic or optical memory including a RAM (volatile and non-volatile), cache, hard drive, or removable storage disk). Communication device 360 can include any suitable device capable of transmitting and receiving signals over a network, such as a network interface chip or device. The components of the computer can be connected in any suitable manner, such as via a wired media (e.g., a physical system bus 380, Ethernet connection, or any other wire transfer technology) or wirelessly (e.g., Bluetooth®, Wi-Fi®, or any other wireless technology).

Software module 350, which can be stored as executable instructions in storage 340 and executed by processor(s) 310, can include, for example, an operating system and/or the processes that embody the functionality of the methods of the present disclosure (e.g., as embodied in the devices as described herein).

Software module 350 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 340, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

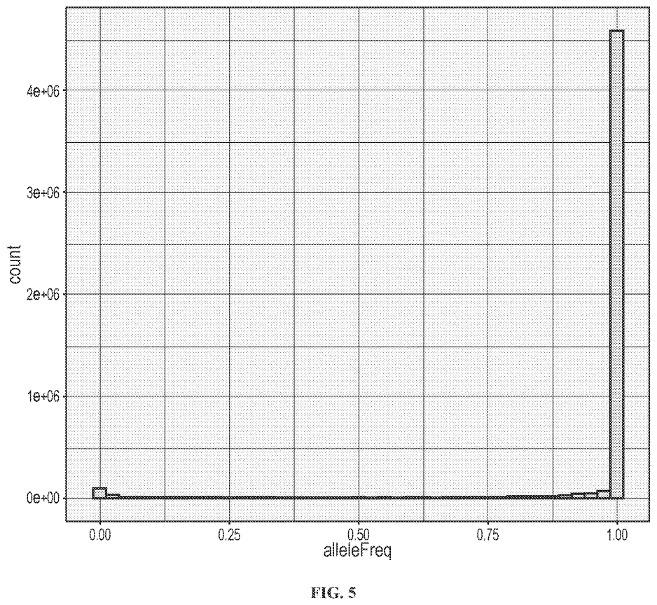

Device 300 may be connected to a network (e.g., network 404, as shown in FIG. 4 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can be implemented using any operating system, e.g., an operating system suitable for operating on the network. Software module 350 can be written in any suitable programming language, such as C, C++, Java, R, or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 310.

Device 300 can further include a sequencer 370, which can be any suitable nucleic acid sequencing instrument.

FIG. 4 illustrates an example of a computing system in accordance with one embodiment. In system 400, device 300 (e.g., as described above and illustrated in FIG. 3) is connected to network 404, which is also connected to device 406. In some embodiments, device 406 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 300 and 406 may communicate, e.g., using suitable communication interfaces via network 404, such as a Local Area Network (LAN), Virtual Private Network (VPN), or the Internet. In some embodiments, network 404 can be, for example, the Internet, an intranet, a virtual private network, a cloud network, a wired network, or a wireless network. Devices 300 and 406 may communicate, in part or in whole, via wireless or hardwired communications, such as Ethernet, IEEE 802.11b wireless, or the like. Additionally, devices 300 and 406 may communicate, e.g., using suitable communication interfaces, via a second network, such as a mobile/cellular network. Communication between devices 300 and 406 may further include or communicate with various servers such as a mail server, mobile server, media server, telephone server, and the like. In some embodiments, Devices 400 and 406 can communicate directly (instead of, or in addition to, communicating via network 404), e.g., via wireless or hardwired communications, such as Ethernet, IEEE 802.11b wireless, or the like. In some embodiments, devices 300 and 406 communicate via communications 408, which can be a direct connection or can occur via a network (e.g., network 404).

One or all of devices 300 and 406 generally include logic (e.g., http web server logic) or are programmed to format data, accessed from local or remote databases or other sources of data and content, for providing and/or receiving information via network 404 according to various examples described herein.

### EXAMPLES

### Example 1—A Non-Limiting Example of Data that Illustrates the Identification of mtDNA Variant Sequences

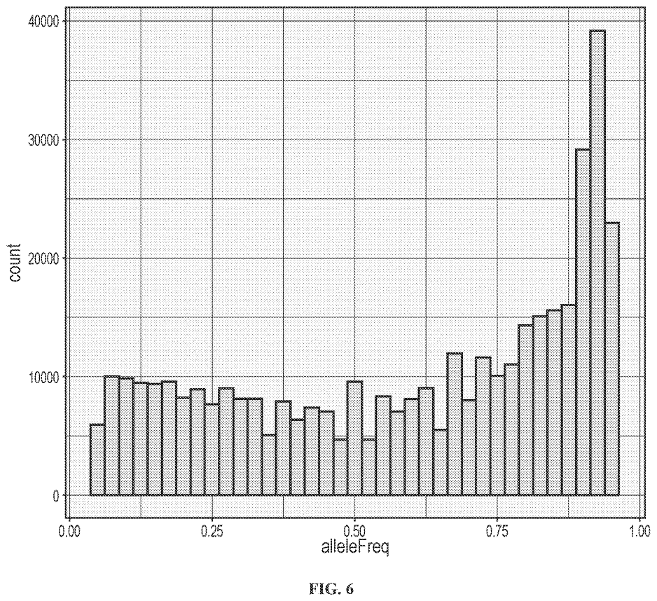

FIG. 5 and FIG. 6 depict non-limiting examples of data that illustrate the identification of mtDNA variant sequences. FIG. 5 depicts a histogram of allele frequencies for mtDNA sequence variants, identified according to methods described in the present disclosure. Such methods can comprise extracting mitochondrial reads from a bam file, realigning the reads to a mitochondrial reference genome (e.g. the revised Cambridge Reference Sequence genome), calling the mitochondrial short variants, creating a mtDNA vcf variants file, providing quality control of the resulting data, and compiling the mtDNA vcf variants file into a final mitochondrial short variants file. 5,198,547 mitochondrial variants were identified in 177, 921 next-generation sequencing samples, and the results are depicted in FIG. 5. In addition, FIG. 5 depicts 4,435,255 (85%) of the variants having an allele frequency of 1.0, suggesting these variants are likely germline. The y-axis of FIG. 5 depicts a raw count of the allele frequencies and the x-axis of FIG. 5 depicts binned mtDNA allele frequencies derived from samples. FIG. 6 depicts the same histogram as FIG. 5, but with a truncated range. FIG. 6 provides a depiction of the histogram for mtDNA allele frequencies greater than 0.05 and less than 0.95. As in FIG. 5, the y-axis of FIG. 6 depicts a raw count of the allele frequencies and the x-axis of FIG. 6 depicts binned mtDNA allele frequencies derived from samples. mtDNA allele frequencies can contribute towards the categorization of a mtDNA sample as germline, somatic, subclonal or an artifact.

### Example 2—A Non-Limiting Example of Data that Illustrates the Use of mtDNA Variant Sequences for Identifying Mitochondrial Haplogroups

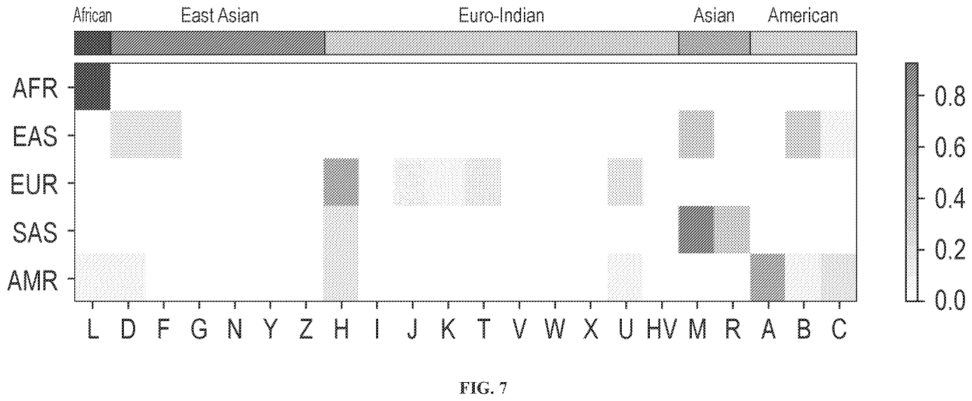

FIG. 7 depicts a non-limiting example of data that illustrates the use of mtDNA variant sequences for identifying mitochondrial haplogroups. The x-axis depicts letter codes for haplogroup categories, where the letter codes relate to human evolution and ancestry.

Certain haplogroups are typically seen in specific ancestries. The y-axis depicts the ancestry calls. FIG. 7 shows the concordance of mtDNA variant-based haplogroups with human ancestral groups. The top bar indicates which haplogroups associated with which human ancestral groups. For example, haplogroup L is associated with African ancestry. The gradient scale bar depicts the percentage of samples from a given ancestry call that had a given independent haplogroup call. For example, >80% of samples with an African ancestry call had an independent L haplogroup call.

### Example 3—A Non-Limiting Example of Data that Illustrates the Use of mtDNA Variant Sequences for Identifying at Least One Associated Disease

Despite the importance of mitochondrial function on the bioenergetics and metabolism of the cell, understanding the functional impact of somatic mitochondrial mutations has been difficult. Additionally, small cohorts, as used in previous studies, lack the power to estimate the prevalence of specific recurrent mutations.

The methods of the present disclosure have been used to evaluate 177540 advance cancer patients, across 84 different tumor types. The patients were sequenced as part of routine clinical care comprising a next-generation sequencing platform. Off-target sequencing reads were mapped to the mitochondrial genome (e.g. the revised Cambridge Reference Sequence), and variant calling was performed. The sequencing methods had a median read depth of 18 and filtering by allele frequency (less than or equal to 95% and greater than or equal to 5%) led to the identification of almost 400,000 predicted somatic mitochondrial variants. Over 80% of patients had at least one mitochondrial variant detected, with an average of 2.2 mitochondrial variants detected per patient. Each recurrent mitochondrial short variant was assessed to identify associations with specific cancers. The alteration with the strongest enrichment was G3224A, a mutation in the mitochondrial leucine tRNA gene (MT-TL1), which had a prevalence of 3.6% (51/1426) in thyroid cancer samples, as compared to 0.05% (85/176114) in all other disease types (OR=76.8, P<2.2e-16). The G3244A mutation had been previously identified as occurring in thyroid cancer, specifically Hurthle cell carcinomas, as well as in the mitochondrial disease MELAS (mitochondrial encephalomyopathy, lactic acidosis, and stroke-like episodes). Thyroid cancer samples in our data with kg3244A were found to be associated with thyroid folloicular carcinomas (7/124, 5.6%) and Hurthle cell carcinomas (7/57, 12.2%). Additional recurrent MT-TL1 mutations were identified in the thyroid cancer cohort at positions 3242 (1426) and 3248 (5/1426).

A mutation in the NADH dehydrogenase subunit MT-ND1 was also identified.

G3380A had been previously shown to occur in thyroid and colorectal cancer, and results in a R25Q mutation that is thought to affect ND1 function. G3380A was identified in 26/1426 (1.8%) of thyroid cancer patients. The majority of G3380A variants occurred in thyroid anaplastic carcinomas 23/26(88.5 %). The dataset derived from methods of the present disclosure has allowed the identification and determination of the prevalence of rare mitochondrial short variant alterations in cancer.

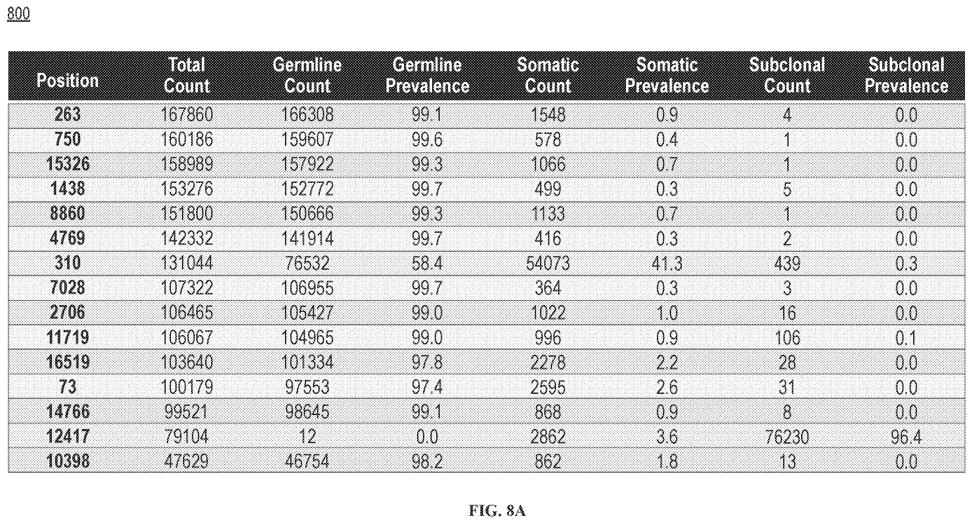

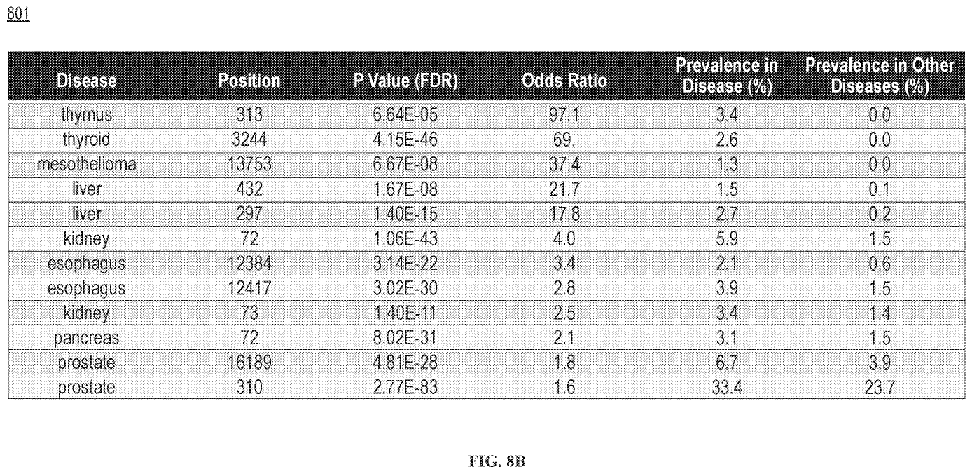

FIG. 8A and FIG. 8B depict non-limiting examples of data in the form of tables that illustrates statistics relating diseases to mtDNA variants. FIG. 8A provides a summary of various mtDNA variants, in terms of the nucleotide position at which the variant occurred, the total count, somatic count, germline count, and subclonal count of the variant, as well as the germline prevalence, somatic prevalence, and subclonal prevalence of the variant. FIG. 8B provides a summary of statistical measures that can depict the nature of the association between a mtDNA variant and a disease, such as, but not limited to, the specificity and strength of the association. The described measures include the name of the disease, namely cancer, the nucleotide position within the mtDNA genome of the mtDNA variant being assessed, the false discovery rate (FDR)-adjusted p-values of the association between the disease and the mtDNA variants being assessed, the odds ratios of the association between the disease and the mtDNA variants being assessed, the prevalence of the mtDNA variant for the corresponding disease, and the prevalence of the mtDNA variant for other diseases.

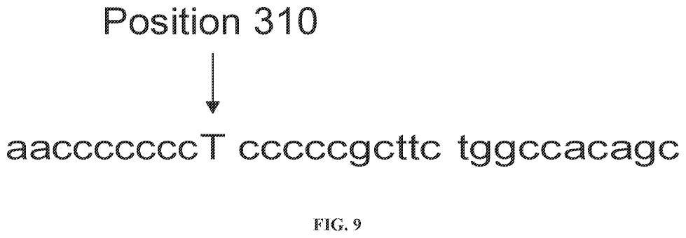

FIG. 9 depicts a non-limiting exemplary schematic that illustrates a wildtype control sequence within the mitochondrial genome. Nucleotide position 310 of the mitochondrial region harbors a single T nucleotide for wildtype sequences, but position 310 can often be the site of polymorphic changes, such as, but not limited to, the addition of a C following the T at position 310, the substitution of a CTC instead of the T at position 310, and the substitution of a CCTC instead of the T at position 310. Variations at position 310 can be very common in disease samples such as, but not limited to, samples deriving from prostate cancer (85% prevalence), non-small cell lung cancer (NSCLC) (79% prevalence), and colorectal cancer (71% prevalence).

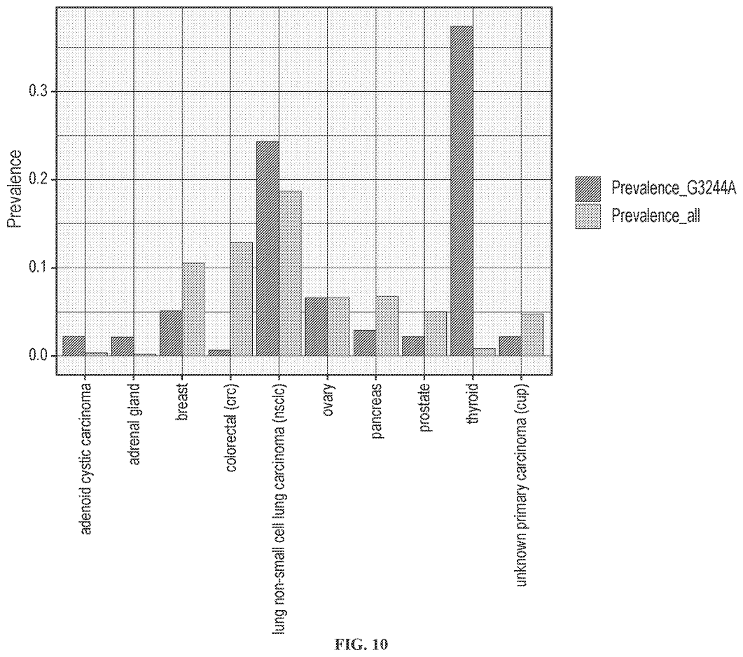

FIG. 10 depicts a non-limiting example of data that illustrates the prevalence of the G3244A mutation as detected in different cancer types (left hand bars in plot) as compared to the prevalence of that cancer in the sample set (right hand bars in plot). For example, breast cancer samples made up ~10% of the dataset, but only 5% of samples with a G3244A variant. 136 samples with the G3244A mutation were detected, of which 37% were thyroid cancer samples and 24% were NSCLC samples. 10 of 11 of the samples with allele frequencies equal to or greater than 0.95 are thyroid cancer samples. One of the samples was an unknown primary sample comprising a tissue thyroid gland. The G3244A mutation is found in a sequence encoding tRNA-L1. The G3244A mutation was previously associated with cancer, including thyroid oncocytoma, gastric carcinoma, and MELAS.

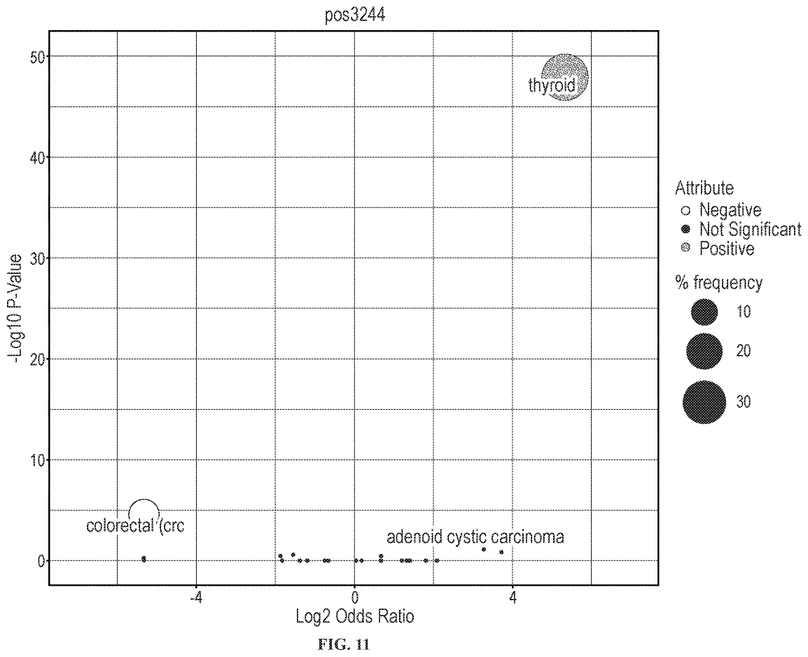

FIG. 11 depicts a non-limiting example of data that illustrates the statistical significance of variants at the 3244 position of the mitochondrial genome, across cancer samples. The depicted data illustrates statistical metrics regarding variants on position 3244 of the mitochondrial genome. The y-axis depicts the negative base 10 logarithm of the p-value, for various disease samples for mtDNA variants on position 3244. The x-axis depicts the base 2 logarithm of the odds ratio, for various disease samples for mtDNA variants on position 3244. The size of each glyph in the figure represents the frequency in percentage of a particular mtDNA variant at position 3244 of the mitochondrial genome. The color of each glyph in the figure represents whether the relationship between the variant and the illness is statistically significant, and if so, the direction of the statistical significance. A strong correlation between a particular mtDNA variant at position 3244 can be observed with thyroid cancer, as depicted by the glyph located on the top right corner of the plot.

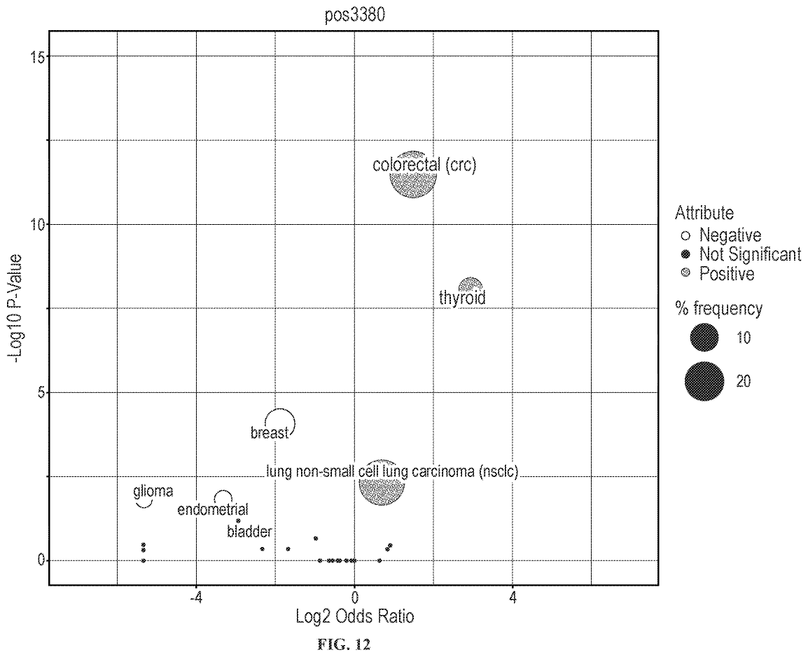

FIG. 12 depicts a non-limiting example of data that illustrates the statistical significance of variants at the 3380 position of the mitochondrial genome, across cancer samples. The depicted data illustrates statistical metrics regarding variants on position 3380 of the mitochondrial genome. The y-axis depicts the negative base 10 logarithm of the p-value, for various disease samples for mtDNA variants on position 3380. The x-axis depicts the base 2 logarithm of the odds ratio, for various disease samples for mtDNA variants on position 3380. The size of each glyph in the figure represents the frequency in percentage of a particular mtDNA variant at position 3380 of the mitochondrial genome. The color of each glyph in the figure represents whether the relationship between the variant and the illness is statistically significant, and if so, the direction of the statistical significance. A correlation between a particular mtDNA variant at position 3380 can be observed with colorectal cancer, as depicted by the glyph located on the upper half of the plot.

### Exemplary Implementations

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, a plurality of off-target sequence reads obtained from a targeted sequencing method performed on the sample from the subject;
   aligning, using the one or more processors, the plurality of off-target sequence reads to a mitochondrial reference genome to identify a plurality of mtDNA alignment reads; and
   processing, using the one or more processors, the plurality of mtDNA alignment reads to identify a mtDNA variant.
2. The method of clause 1, wherein a variant caller is used to process the plurality of mtDNA alignment reads and identify the mtDNA variant.
3. The method of clause 1, further comprising:
   determining, using the one or more processors, an allele frequency for the mtDNA variant based on the plurality of mtDNA alignment reads; and
   categorizing, using the one or more processors, the mtDNA variant as germline, somatic, or as subclonal or an artifact, based on the determined allele frequency.
4. The method of clause 3, wherein the categorization does not require use of sequence read data from a matched normal control.
5. The method of any of clauses 1 to 4, wherein the subject is suspected of having or is determined to have cancer.
6. The method of clause 5, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
7. The method of clause 5, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
8. The method of clause 7, further comprising treating the subject with an anti-cancer therapy.
9. The method of clause 8, wherein the anti-cancer therapy comprises a targeted anti-cancer therapy.
10. The method of clause 9, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
11. The method of any one of clauses 1 to 10, further comprising obtaining the sample from the subject.
12. The method of any one of clauses 1 to 11, wherein the sample comprises a tissue biopsy sample, a liquid biopsy sample, or a normal control.
13. The method of clause 12, wherein the sample is a liquid biopsy sample and comprises blood, plasma, cerebrospinal fluid, sputum, stool, urine, or saliva.
14. The method of clause 12, wherein the sample is a liquid biopsy sample and comprises circulating tumor cells (CTCs).
15. The method of clause 12, wherein the sample is a liquid biopsy sample and comprises cell-free DNA (cfDNA), circulating tumor DNA (ctDNA), or any combination thereof.
16. The method of any one of clauses 1 to 15, wherein the plurality of nucleic acid molecules comprises a mixture of tumor nucleic acid molecules and non-tumor nucleic acid molecules.
17. The method of clause 16, wherein the tumor nucleic acid molecules are derived from a tumor portion of a heterogeneous tissue biopsy sample, and the non-tumor nucleic acid molecules are derived from a normal portion of the heterogeneous tissue biopsy sample.
18. The method of clause 16, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
19. The method of any one of clauses 1 to 18, wherein the one or more adapters comprise amplification primers, flow cell adaptor sequences, substrate adapter sequences, or sample index sequences.
20. The method of any one of clauses 1 to 19, wherein the captured nucleic acid molecules are captured from the amplified nucleic acid molecules by hybridization to one or more bait molecules.
21. The method of clause 20, wherein the one or more bait molecules comprise one or more nucleic acid molecules, each comprising a region that is complementary to a region of a captured nucleic acid molecule.
22. The method of any one of clauses 1 to 21, wherein amplifying nucleic acid molecules comprises performing a polymerase chain reaction (PCR) amplification technique, a non-PCR amplification technique, or an isothermal amplification technique.
23. The method of any one of clauses 1 to 22, wherein the sequencing comprises use of a massively parallel sequencing (MPS) technique, whole genome sequencing (WGS), whole exome sequencing, targeted sequencing, direct sequencing, or Sanger sequencing technique.
24. The method of clause 23, wherein the sequencing comprises massively parallel sequencing, and the massively parallel sequencing technique comprises next generation sequencing (NGS).
25. The method of any one of clauses 1 to 24, wherein the sequencer comprises a next generation sequencer.
26. The method of any one of clauses 1 to 25, wherein one or more of the plurality of sequencing reads overlap one or more gene loci within one or more subgenomic intervals in the sample.
27. The method of clause 26, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
28. The method of clause 26 or clause 27, wherein the one or more gene loci comprise ABL 1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74,CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4(C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
29. The method of clause 26 or clause 27, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K⊕, PIGF, PTCH, RAF, RANKL, RET, ROS1, SLAMF7, VEGF, VEGFA, VEGFB, or any combination thereof.
30. The method of any one of clauses 1 to 29, further comprising generating, by the one or more processors, a report indicating the presence or absence of the genetic variant.
31. The method of clause 30, further comprising transmitting the report to a healthcare provider.
32. The method of clause 31, wherein the report is transmitted via a computer network or a peer-to-peer connection.
33. A method for identifying mitochondrial DNA (mtDNA) variants, comprising:
   a) receiving, at one or more processors, a plurality of off-target sequence reads obtained from a targeted sequencing method performed on a sample from a subject;
   b) aligning, using the one or more processors, the plurality of off-target sequence reads to a mitochondrial reference genome to identify a plurality of mtDNA alignment reads; and
   c) processing, using the one or more processors, the plurality of mtDNA alignment reads to identify a mtDNA variant.
34. The method of clause 33, wherein a variant caller is used to process the plurality of mtDNA alignment reads and identify the mtDNA variant.
35. The method of clause 34, further comprising:
   d) determining, using the one or more processors, an allele frequency for the mtDNA variant based on the plurality of mtDNA alignment reads; and
   e) categorizing, using the one or more processors, the mtDNA variant as germline, somatic, or as subclonal or an artifact, based on the determined allele frequency.
36. The method of clause 35, wherein the categorization does not require use of sequence read data from a matched normal control.
37. The method of any of clauses 33 to 36, wherein the off-target sequence reads are identified by aligning sequence reads from the targeted sequencing method to a non-mitochondrial reference genome.
38. The method of clause 37, wherein the non-mitochondrial reference genome is a human reference genome.
39. The method of clause 38, wherein the human reference genome is hg19.
40. The method of any of clauses 33 to 39, wherein the off-target sequence reads comprise sequence reads that align to a mitochondrial genome and sequence reads that do not align to a non-mitochondrial reference genome.
41. A method for identifying mtDNA variants, comprising:
   a) receiving, at one or more processors, a plurality of sequence reads obtained from a targeted sequencing method performed on a sample from a subject;
   b) aligning, using the one or more processors, the plurality of sequence reads to a mitochondrial reference genome, to identify a plurality of mtDNA alignment reads;
   c) processing, using the one or more processors, the plurality of mtDNA alignment reads to identify a mtDNA variant;
   d) determining, using the one or more processors, an allele frequency for the mtDNA variant based on the plurality of mtDNA alignment reads; and
   e) categorizing, using the one or more processors, the mtDNA variant as germline, somatic, or as subclonal or an artifact, based on the determined allele frequency, wherein the categorization does not require use of sequence read data from a matched normal control.
42. The method of clause 41, wherein a variant caller is used to process the plurality of mtDNA alignment reads and identify the mtDNA variant.
43. The method of any of clauses 1 to 42, wherein the mitochondrial reference genome is the revised Cambridge Reference Sequence.
44. The method of any of clauses 2 to 43, wherein the variant caller comprises a short variant caller.
45. The method of any of clauses 2 to 44, wherein the variant caller comprises a mtDNA rearrangement caller.
46. The method of any of clauses 2 to 45, wherein the variant caller comprises a large mtDNA deletion caller.
47. The method of any of clauses 33 to 46, wherein a mtDNA variant is classified as germline if its allele frequency is greater than or equal to a first threshold.
48. The method of clause 47, wherein the first threshold is about 90%, 95% or 98%.
49. The method of clause 46 or clause 47, wherein a mtDNA variant is classified as somatic if its allele frequency is less than the first threshold and greater than or equal to a second threshold.
50. The method of clause 49, wherein the second threshold is about 2%, 5%, or 10%.
51. The method of any of clauses 49 to 50, wherein a mtDNA variant is classified as subclonal or as an artifact if its allele frequency is less than the second threshold.
52. The method of clause 51, wherein the first threshold is 95%, and the second threshold is 5%
53. The method of any of clauses 33 to 52, wherein mtDNA variant allele frequencies are used to identify a mitochondrial haplogroup.
54. The method of clause 53, wherein the mitochondrial haplogroup is used to determine ancestral data regarding the subject.
55. A method for detection of a disease in a subject, the method comprising:
   a) receiving, at one or more processors, a plurality of off-target sequence reads obtained from a targeted sequencing method performed on a sample from the subject;
   b) aligning, using the one or more processors, the plurality of sequence reads to a mitochondrial reference genome to identify a plurality of mtDNA alignment reads;
   c) processing, using the one or more processors, the plurality of mtDNA alignment reads using a variant caller to identify a mtDNA variant; and
   d) detecting, using the one or more processors, a disease signal in the subject based on at least one identified mtDNA variant, wherein the disease signal is indicative of a disease status of the subject.
56. The method of clause 55, wherein the disease comprises a thyroid cancer and the mtDNA variant is a G3244A substitution in a mitochondrial leucine tRNA (MT-TL1) gene.
57. The method of clause 56, wherein the thyroid cancer is Hurthle cell carcinoma.
58. The method of clause 55, wherein the disease comprises mitochondrial encephalomyopathy, lactic acidosis, and stroke-like episodes (MELAS) and the mtDNA variant is a G3244A substitution.
59. The method of clause 55, wherein the disease comprises a thyroid cancer and the mtDNA variant is a G3380A substitution in a NADH dehydrogenase (ND1) gene.
60. The method of clause 59, wherein the thyroid cancer is thyroid anaplastic carcinoma.
61. The method of clause 55, wherein the disease comprises a colorectal cancer and the mtDNA variant is a G3380A substitution in a NADH dehydrogenase (ND1) gene.
62. The method of clause 55, wherein the disease comprises MELAS and the mtDNA variant is a G3380A substitution.
63. The method of any of clauses 33 to 62, wherein the subject is a human.
64. The method of any of clauses 33 to 63, wherein the sample comprises a tissue biopsy sample.
65. The method of any of clauses 33 to 64, wherein the sample comprises a liquid biopsy sample.
66. The method of clause 65, wherein the liquid biopsy sample comprises blood, plasma, cerebrospinal fluid, sputum, stool, urine, or saliva.
67. A method for diagnosing a disease, the method comprising diagnosing that a disease based on an identification of a mtDNA variant for a sample from the subject, wherein the mtDNA variant is determined according to the method of any one of clauses 1 to 66.
68. A method of selecting an anti-cancer therapy, the method comprising: responsive to identifying a mtDNA variant for a sample from a subject, selecting an anti-cancer therapy for the subject, wherein identifying a mtDNA variant is determined according to the method of any one of clauses 1 to 67.
69. A method of treating a cancer in a subject, comprising: responsive to identifying a mtDNA variant for a sample from the subject, administering an effective amount of an anti-cancer therapy to the subject, wherein identifying a mtDNA variant is determined according to the method of any one of clauses 1 to 68.
70. A method for monitoring cancer progression or recurrence in a subject, the method comprising:
   determining a first identification of a mtDNA variant in a first sample obtained from the subject at a first time point according to the method of any one of clauses 1 to 69;
   determining a second identification of a mtDNA variant in a second sample obtained from the subject at a second time point; and comparing the first mtDNA variant to the second mtDNA variant, thereby monitoring the cancer progression or recurrence.
71. The method of clause 70, wherein the second mtDNA variant for the second sample is determined according to the method of any one of clauses 1 to 70.
72. The method of clause 70 or clause 71, further comprising selecting an anti-cancer therapy for the subject in response to the cancer progression.
73. The method of clause 70 or clause 71, further comprising administering an anti-cancer therapy to the subject in response to the cancer progression.
74. The method of clause 70 or clause 71, further comprising adjusting an anti-cancer therapy for the subject in response to the cancer progression.
75. The method of any one of clauses 72 to 74, further comprising adjusting a dosage of the anti-cancer therapy or selecting a different anti-cancer therapy in response to the cancer progression.
76. The method of clause 75, further comprising administering the adjusted anti-cancer therapy to the subject.
77. The method of any one of clauses 70 to 76, wherein the first time point is before the subject has been administered an anti-cancer therapy, and wherein the second time point is after the subject has been administered the anti-cancer therapy.
78. The method of any one of clauses 70 to 77, wherein the subject has a cancer, is at risk of having a cancer, is being routine tested for cancer, or is suspected of having a cancer.
79. The method of any one of clauses 70 to 78, wherein the cancer is a solid tumor. 80. The method of any one of clauses 70 to 78, wherein the cancer is a hematological cancer.
81. The method of any one of clauses 72 to 80, wherein the anti-cancer therapy comprises chemotherapy, radiation therapy, immunotherapy, a targeted therapy, or surgery.
82. The method of any one of clauses 1 to 81, further comprising determining, identifying, or applying the mtDNA variant for the sample as a diagnostic value associated with the sample.
83. The method of any one of clauses 1 to 82, further comprising generating a genomic profile for the subject based on the identification of a mtDNA variant.
84. The method of clause 83, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
85. The method of clause 83 or clause 84, wherein the genomic profile for the subject further comprises results from a nucleic acid sequencing-based test.
86. The method of any one of clauses 83 to 85, further comprising selecting an anti-cancer therapy, administering an anti-cancer therapy, or applying an anti-cancer therapy to the subject based on the generated genomic profile.
87. The method of any one of clauses 1 to 86, wherein the identification of an mtDNA variant for the sample is used in making suggested treatment decisions for the subject.
88. The method of any one of clauses 1 to 87, wherein the identification of an mtDNA variant for the sample is used in applying or administering a treatment to the subject.
89. A system comprising:
   a) one or more processors; and
   b) a memory communicatively coupled to the one or more processors and configured to store instructions that, when executed by the one or more processors, cause the system to:
      i. receive a plurality of off-target sequence reads obtained from a targeted sequencing method performed on a sample from a subject;
      ii. align the plurality of off-target sequence reads to a mitochondrial reference genome to identify a plurality of mtDNA alignment reads; and
      iii. process the plurality of mtDNA alignment reads to identify a mtDNA variant.
90. The system of clause 89, wherein a variant caller is used to process the plurality of mtDNA alignment reads and identify the mtDNA variant.
91. The system of clause 89 or clause 90, wherein the off-target sequence read data are identified by aligning sequence reads from a targeted sequencing method to a non-mitochondrial reference genome.
92. The system of any of clauses 89 to 91, wherein the off-target sequence reads comprise sequence reads that align to a mitochondrial genome and sequence reads that do not align to a non-mitochondrial reference genome.
93. The system of any of clauses 89 to 92, wherein the variant caller comprises a short variant caller.
94. The system of any of clauses 89 to 93, further comprising instructions that, when executed by the one or more processors, cause the system to:
   c) determine an allele frequency for the mtDNA variant based on the plurality of mtDNA alignment reads; and
   d) categorize the mtDNA variant as germline, somatic, or as subclonal or an artifact, based on the determined allele frequency.
95. The system of clause 94, wherein the categorization step does not require use of sequence read data from a matched normal control.
96. A non-transitory computer-readable storage medium storing one or more programs, the one or more programs comprising instructions, which when executed by one or more processors of a system, cause the system to:
   a) receive a plurality of off-target sequence reads obtained from a targeted sequencing method performed on a sample from a subject;
   b) align the plurality of sequence reads to a mitochondrial reference genome to identify a plurality of mtDNA alignment reads; and
   c) process the plurality of mtDNA alignment reads to identify a mtDNA variant.
97. The non-transitory computer-readable storage medium of clause 96, wherein a variant caller is used to process the plurality of mtDNA alignment reads and identify the mtDNA variant.
98. The non-transitory computer-readable storage medium of clause 96 or clause 97, wherein the off-target sequence reads are identified by aligning sequence reads from a targeted sequencing method to a non-mitochondrial reference genome.
99. The non-transitory computer-readable storage medium of any of clauses 96 to 98, wherein the off-target sequence reads comprise sequence reads that align to a mitochondrial genome and sequence reads that do not align to a non-mitochondrial reference genome.
100. The non-transitory computer-readable storage medium of any of clauses 96 to 99, wherein the variant caller comprises a short variant caller. 101. The non-transitory computer-readable storage medium of any of clauses 96 to 100, further comprising instructions that, when executed by the one or more processors, cause the system to:
   d) determine an allele frequency for the mtDNA variant based on the plurality of mtDNA alignment reads; and
   e) categorize the mtDNA variant as germline, somatic, or as subclonal or an artifact, based on the determined allele frequency.
102. The non-transitory computer-readable storage medium of clause 101, wherein the categorization step does not require use of sequence read data from a matched normal control.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, a plurality of off-target sequence reads obtained from a targeted sequencing method performed on the sample from the subject;
aligning, using the one or more processors, the plurality of off-target sequence reads to a mitochondrial reference genome to identify a plurality of mtDNA alignment reads; and
processing, using the one or more processors, the plurality of mtDNA alignment reads to identify a mtDNA variant.

2. The method of claim 1, wherein a variant caller is used to process the plurality of mtDNA alignment reads and identify the mtDNA variant.

3. The method of claim 2, further comprising:
determining, using the one or more processors, an allele frequency for the mtDNA variant based on the plurality of mtDNA alignment reads; and
categorizing, using the one or more processors, the mtDNA variant as germline, somatic, or as subclonal or an artifact, based on the determined allele frequency.

4. (canceled)

5. The method of claim 1, wherein the off-target sequence reads are identified by aligning sequence reads from the targeted sequencing method to a non-mitochondrial reference genome.

6. (canceled)

7. The method of claim 1, wherein the off-target sequence reads comprise sequence reads that align to a mitochondrial genome and sequence reads that do not align to a non-mitochondrial reference genome.

8. A method for identifying mtDNA variants, comprising:
a) receiving, at one or more processors, a plurality of sequence reads obtained from a targeted sequencing method performed on a sample from a subject;
b) aligning, using the one or more processors, the plurality of sequence reads to a mitochondrial reference genome, to identify a plurality of mtDNA alignment reads;
c) processing, using the one or more processors, the plurality of mtDNA alignment reads to identify a mtDNA variant;
d) determining, using the one or more processors, an allele frequency for the mtDNA variant based on the plurality of mtDNA alignment reads; and
e) categorizing, using the one or more processors, the mtDNA variant as germline, somatic, or as subclonal or an artifact, based on the determined allele frequency, wherein the categorization does not require use of sequence read data from a matched normal control.

9. The method of claim 8, wherein a variant caller is used to process the plurality of mtDNA alignment reads and identify the mtDNA variant.

10. (canceled)

11. The method of claim 2, wherein the variant caller comprises a short variant caller, a mtDNA rearrangement caller, or a large mtDNA deletion caller.

12. The method of claim 1, wherein a mtDNA variant is classified as germline if its allele frequency is greater than or equal to a first threshold.

13. (canceled)

14. The method of claim 12, wherein a mtDNA variant is classified as somatic if its allele frequency is less than the first threshold and greater than or equal to a second threshold.

15. (canceled)

16. The method of claim 14, wherein a mtDNA variant is classified as subclonal or as an artifact if its allele frequency is less than the second threshold.

17. (canceled)

18. The method of claim 1, wherein mtDNA variant allele frequencies are used to identify a mitochondrial haplogroup.

19. The method of claim 18, wherein the mitochondrial haplogroup is used to determine ancestral data regarding the subject.

20. A method for detection of a disease in a subject, the method comprising:
a) receiving, at one or more processors, a plurality of off-target sequence reads obtained from a targeted sequencing method performed on a sample from the subject;
b) aligning, using the one or more processors, the plurality of sequence reads to a mitochondrial reference genome to identify a plurality of mtDNA alignment reads;
c) processing, using the one or more processors, the plurality of mtDNA alignment reads using a variant caller to identify a mtDNA variant; and
d) detecting, using the one or more processors, a disease signal in the subject based on at least one identified mtDNA variant, wherein the disease signal is indicative of a disease status of the subject.

21. The method of claim 20, wherein the disease comprises a thyroid cancer and the mtDNA variant is a G3244A substitution in a mitochondrial leucine tRNA (MT-TL1) gene.

22. The method of claim 21, wherein the thyroid cancer is Hurthle cell carcinoma.

23. The method of claim 20, wherein the disease comprises mitochondrial encephalomyopathy, lactic acidosis, and stroke-like episodes (MELAS) and the mtDNA variant is a G3244A substitution.

24. The method of claim 20, wherein the disease comprises a thyroid cancer and the mtDNA variant is a G3380A substitution in a NADH dehydrogenase (ND1) gene.

25. (canceled)

26. The method of claim 20, wherein the disease comprises a colorectal cancer and the mtDNA variant is a G3380A substitution in a NADH dehydrogenase (ND1) gene.

27. The method of claim 20, wherein the disease comprises MELAS and the mtDNA variant is a G3380A substitution.

28. (canceled)

29. (canceled)

30. (canceled)
